(19) United States
(12) Patent Application Publication
(10) Pub. No.: US 2025/0295779 A1
(43) Pub. Date: Sep. 25, 2025

(54) BIO-NANOSHELLS AND METHODS FOR PREPARATION AND APPLICATIONS THEREOF

(71) Applicants: Emily Shannon Day, Landenburg, PA (US); Sara Aboeleneen, Newark, DE (US)

(72) Inventors: Emily Shannon Day, Landenburg, PA (US); Sara Aboeleneen, Newark, DE (US)

(73) Assignee: University of Delaware, Newark, DE (US)

(21) Appl. No.: 19/086,533

(22) Filed: Mar. 21, 2025

Related U.S. Application Data
(60) Provisional application No. 63/568,293, filed on Mar. 21, 2024.

(51) Int. Cl. A61K 41/00; A61K 9/51; A61P 35/00

(52) U.S. Cl. CPC A61K 41/0052; A61K 9/5115; A61K 9/5176; A61K 9/5192; A61P 35/00

(57) ABSTRACT

The present invention provides a bio-nanoshell for binding specifically to a target cell. The bio-nanoshell may comprise a cell-derived biological membrane from a donor cell and a nanoshell having an exterior surface coated with the cell-derived biological membrane. The cell-derived biological membrane may comprise a phospholipid bilayer and an adhesion protein specific for the target cell. Also provided is a method for preparing the bio-nanoshells and a method for delivering the bio-nanoshells to target cells.

### REFERENCE TO U.S. GOVERNMENT SUPPORT

This invention was made with government support under grant number DMR1752009 awarded by the National Science Foundation (NSF) and grant number RO1CA211925 awarded by the National Institutes of Health (NIH). The United States has certain rights in the invention.

This application claims priority to U.S. Provisional Application No. 63/568,293, filed Mar. 21, 2024, and the contents of which are incorporated herein by reference in their entireties for all purposes.

### FIELD OF THE INVENTION

The invention relates to bio-nanoshells for delivery to target cells, methods of making the same, and uses thereof.

### BACKGROUND OF THE INVENTION

Light-responsive nanoparticles (NP) that emit heat upon excitation with light can be utilized in numerous biomedical applications to enable imaging and/or treatment of disease. “Nanoshells” are one such form of light-responsive nanoparticle. The success of these nanoparticles relies on sufficient binding to and/or uptake by diseased cells in vitro or in vivo. Traditionally, nanoshells and other light-responsive NPs are coated with poly(ethylene glycol) (PEG) to minimize protein opsonization and extend circulation in the blood, but PEG coatings do not enable specific targeting of diseased cells and they can cause an undesirable immune response that triggers the accelerated blood clearance phenomenon, in which second doses of PEG-coated NPs are rapidly cleared from circulation. Nanoshells and other light-responsive NPs have also been coated with molecules such as antibodies, peptides, or aptamers to enable cell-specific targeting, but these approaches have only modestly improved delivery compared to PEG-coated NPs.

There remains a critical need for surface modifications that can elevate nanoshell delivery to diseased cells or tissues while minimizing delivery to non-targeted cells or tissues. By increasing the amount of nanoshells delivered to diseased cells and tissues, greater imaging contrast enhancement and/or greater phototherapeutic outcomes can be achieved.

### SUMMARY OF THE INVENTION

The present invention relates to bio-nanoshells, methods of preparation, and delivery thereof to target cells. The inventors have surprisingly discovered that a new method to coat nanoshells with cell-derived biological membranes enables the nanoshells to better target diseased versus non-diseased cells in vitro and to better evade the immune system and target diseased cells/tissues in vivo.

A bio-nanoshell for binding specifically to a target cell is provided. The bio-nanoshell comprises a cell-derived biological membrane from a donor cell and a nanoshell having an exterior surface coated with the cell-derived biological membrane, wherein the cell-derived biological membrane comprises a phospholipid bilayer and an adhesion protein specific for the target cell. The bio-nanoshell may be photothermal. The target cell may be a cancer cell.

A method for preparing bio-nanoshells capable of binding specifically to target cells is provided. The preparation method comprises: (a) combining nanoshells with cell-derived biological membranes from donor cells, wherein each cell-derived biological membrane comprises a phospholipid bilayer and an adhesion protein specific for the target cells; and (b) coating an exterior surface of each of the nanoshells with one of the cell-derived biological membranes, whereby bio-nanoshells are prepared.

The preparation method may further comprise extracting the cell-derived biological membranes from the donor cells, whereby extracted cell-derived biological membranes are generated. The delivery method may further comprise extruding the extracted cell-derived biological membranes through a porous extruder filter at a temperature of 80-90° C., whereby membrane vesicles of the cell-derived biological membrane are formed. The delivery method may further comprise combining the cell-derived biological membranes with the nanoshells at a ratio between 20:1 and 1:20. The delivery method may further comprise co-extruding the cell-derived biological membranes and the nanoshells through a porous extruder filter at a temperature of 80-90° C.

For each preparation method, bio-nanoshells prepared according to the method are provided.

A method for delivering bio-nanoshells to target cells is provided. The delivery method comprises: (a) exposing the target cells to bio-nanoshells, wherein each of the bio-nanoshells comprises a nanoshell having an exterior surface coated with a cell-derived biological membrane from a donor cell, wherein each of the cell-derived biological membranes comprises a phospholipid bilayer and an adhesion protein specific for one of the target cells; and (b) binding the bio-nanoshells specifically to the target cells via the cell-derived biological membranes.

According to the preparation method, the target cells may be in a subject. The donor cells may be from the subject. The delivery method may further comprise administering the bio-nanoshell to the subject.

Where the bio-nanoshells are photothermal, the delivery method may further comprise applying light to the bio-nanoshells. The delivery method further comprise emitting heat by the bio-nanoshells. The delivery method may further comprise killing the target cells by the heat. The delivery method may further comprise altering a function of the target cells by the heat. Where the target cells are in a subject suffering from a disease or condition, the delivery method may further comprise treating the disease or condition with the heat.

The delivery method may further comprise emitting photoluminescence or scattering light by the bio-nanoshells.

Where the target cells are in a biological tissue, the delivery method may further comprise generating an acoustic wave by the biological tissue.

### BRIEF DESCRIPTION OF THE DRAWINGS

FIG. 1 shows a scheme of the synthesis of membrane-wrapped nanoshells. Corresponding transmission electron micrographs of uncoated (bare) nanoshells, extruded cell membrane vesicles, and membrane-wrapped nanoshells are shown. Created with BioRender.com.

FIGS. 2A-2B show characterization of membrane-wrapped nanoshells (MWNS). (A) Hydrodynamic diameter and zeta potential of uncoated nanoshells (Bare NS), extruded 4T1 cell membrane vesicles (MV), and MWNS (data show mean and standard deviation, n=3). (B) SDS-PAGE gel showing proteins from (I) known ladder, (II) 4T1 whole cell lysate, (III) extruded 4T1 cell membrane vesicles, (IV) MWNS, and (V) nuclear pellet. Red rectangles denote bands that are present in the whole cell lysate and nuclear fraction lanes but diminished in the membrane vesicle and MWNS lanes, indicating the removal of nuclear proteins from the extracted cell membranes and MWNS. Similar bands for the 4T1 MVs and 4T1 MWNS suggest membrane proteins are maintained during wrapping.

FIGS. 3A-3C show characterization of the optical properties of MWNS. (A) UV-visible extinction profile of Bare NS and MWNS. (B) Temperature of Bare NS and MWNS (diluted in water to a concentration of 10¹⁰ NS/mL) under 808 nm laser irradiation at 1.5 W/cm² (n=3). (C) Photoacoustic (PA) signal generated from MWNS diluted in water at different optical densities ranging from 0 to 6 (n=3). All error bars represent standard deviations.

FIG. 4 shows cytocompatibility of MWNS. Metabolic activity of 4T1 cells treated with MWNS or PEG-NS for 4 h at different optical densities (OD) as measured by AlamarBlue assay. Data are normalized to the metabolic activity of untreated cells (n=3). Error bars represent standard deviations.

FIGS. 5A-5C demonstrate in vitro targeting to homotypic cells. (A) Inductively coupled plasma-mass spectrometry (ICP-MS) measurement of gold content in 4T1 cells or EpH4-Ev cells treated with MWNS for 4 h. The elevated gold content in 4T1 cells indicates MWNS preferentially target homotypic 4T1 cells (n=3). *indicates p<0.05 by Student's t-test. (B) ICP-MS quantification and (C) multiphoton microscopy visualization of NS in 4T1 cells after 6 h incubation with MWNS or PEG-coated NS (n=3). Both ICP-MS and multiphoton microscopy show that 4T1 cells preferentially interact with MWNS as compared to PEG-NS. In microscopy images, cyan=NS photoluminescence and red=cell membrane labeled with CellVue Membrane Dye. Scale bars=25 μm. In ICP-MS data, *indicates p<0.05 by one-way ANOVA with post hoc Tukey. All error bars represent standard deviations.

FIG. 6 shows in vitro evaluation of photothermal therapy mediated by MWNS. Live/dead staining of 4T1 cells after they were incubated with MWNS, PEG-NS, or saline for 4 h and then irradiated in the center of the well with an 808 nm laser at 60 W/cm² for 2 min to induce NS heating. Green indicates live cells (Calcein AM) and red indicates dead cells (Ethidium homodimer-III). White dotted circle indicates the irradiated region of the well. In the absence of light exposure (top row), both PEG-NS and MWNS do not induce cell death. When irradiated with light (bottom row), the MWNS induce greater cell death than PEG-NS.

FIGS. 7A-7C show in vitro evaluation of photoacoustic (PA) imaging contrast enabled by MWNS. (A) Scheme of phantoms containing 4T1 cells that were labeled with MWNS or PEG-NS and used for PA imaging. Created with BioRender.com (B) Combined ultrasound (US; gray scale) and photoacoustic (PA; red scale) overlay images of dome-shaped phantoms containing 4T1 cells labeled with MWNS, PEG-NS, or saline. Scale bars=1 mm. (C) Corresponding quantification of PA signal amplitudes at 680 nm, showing mean PA signal is significantly higher in 4T1 cells exposed to 4T1 MWNS as compared to PEG-NS or saline (n=3). *indicates p<0.05 by one-way ANOVA with post hoc Tukey. Error bars represent standard deviations.

FIGS. 8A-8D show that MWNS can accumulate in tumors to enhance photoacoustic imaging contrast. (A) Scheme of experimental design to evaluate MWNS or PEG-NS delivery to 4T1 tumors in mice after intravenous administration and their use as PA imaging contrast agents. Created with BioRender.com. (B) Ultrasound (gray scale) and photoacoustic (green scale) images of murine 4T1 tumors (outlined in red) at baseline (pre-injection) and 6 hours after intravenous injection of MWNS or PEG-NS. Scale bars=0.5 mm. (C) Quantification of the change in tumor photoacoustic signal intensity from pre-injection baseline to 6 h post-injection for mice treated with MWNS or PEG-NS (n=5). *p<0.05 by student's t-test. Error bars represent standard error of the mean. (D) Representative silver-stained tumor sections from mice that received saline, MWNS, or PEG-NS 24 h post injection show increased MWNS accumulation in the tumor. Silver stain nucleates on the gold surface of NS to enable visualization by light microscopy. Sections were counterstained with hematoxylin to label cell nuclei. (Grey/black dots/regions=NS, purple/blue regions=cell nuclei). Scale bars=75 μm.

FIGS. 9A-9C show phototherapeutic effect of MWNS in vivo. (A) Scheme of experimental design to test the ability of MWNS to mediate photothermal therapy of tumors. Created with BioRender.com. (B) Relative tumor volume of mice treated with MWNS+light, PEG-NS+light, or saline+light. *p<0.05 by ANOVA with post hoc Tukey. Error bars represent standard error of the mean. (C) Representative Ki67 stained tumor sections from mice treated with saline±light, PEG-NS±light, or MWNS±light. Hematoxylin=purple; Ki67=maroon/brown. Scale bars=150 μm. The average percent of Ki67 positive cells was quantified from 7 distinctive tumor regions per mouse using QuPath software. **indicates p<0.01 as determined by one-way ANOVA with post hoc Tukey. Error bars represent standard error of the mean.

FIGS. 10A-10B evaluate the safety of MWNS. (A) Representative images of hematoxylin and eosin (H&E)-stained livers and spleens from mice that received saline±light, PEG-NS±light and MWNS±light. MWNS and PEG-NS do not alter the histological appearance of major clearance organs. Scale bars=275 μm. (B) Mouse weight versus time in each treatment group.

### DETAILED DESCRIPTION OF THE INVENTION

The present invention relates to a new hybrid technology consisting of “nanoshells” that are wrapped with cell-derived biological membranes, forming bio-nanoshells (BioNS) (also known as membrane-wrapped nanoshells, or MWNS) that enable specific recognition of targeted diseased cells to act as imaging contrast agents and/or phototherapeutic agents. Previously, researchers have used PEG- or ligand-coated nanoshells to photothermally treat and/or image various diseases, but these nanoshells enter both targeted and non-targeted cells in vitro and distribute nonspecifically throughout the body with relatively low disease site-accumulation in vivo. This poor delivery efficiency leads to insufficient phototherapeutic and diagnostic effect. The present invention is based on the inventors' surprising discovery of a new method to coat nanoshells with cell-derived biological membranes that enables the nanoshells to better target diseased versus non-diseased cells in vitro and to better evade the immune system and target diseased cells/tissues in vivo. In turn, this improved delivery enables more successful imaging and phototherapy. Notably, the specific cell-derived biological membrane used to produce BioNS could be altered to target and thus image and/or treat a multitude of diseases.

The commercial relevance of this invention is tremendous. These cell-mimetic BioNS could be applied for imaging and/or treatment of various cancer types (e.g. breast, prostate, head and neck, colorectal, brain, liver, pancreatic, melanoma, ovarian, esophageal, and lung cancer) in either human or veterinary subjects. Expanding beyond cancer, nanoshells coated with distinct membrane types could be used to manage other conditions including but not limited to endometriosis, fibrosis, cardiovascular diseases (e.g., atherosclerosis), bacterial or fungal infections, wounds, skin conditions (e.g., psoriasis, eczema, vitiligo, acne, scarring), chronic pain (e.g., pelvic pain or pain associated with musculoskeletal disorders such as osteoarthritis, rheumatoid arthritis, and carpal tunnel syndrome), periodontal/gum diseases, ophthalmic diseases (e.g. age-related macular degeneration), and non-malignant tumors. BioNS could be produced using membranes sourced from cell lines or primary cells obtained from a human or veterinary subject. BioNS could also be produced using a patient's own cells (extracted through a biopsy), thereby allowing this invention to enable making a personalized treatment to minimize or avoid an immune response.

The term “nanoshell” refers to a specific type of spherical nanoparticle comprising a dielectric core (e.g., a 120-130 nm diameter silica sphere) surrounded by a metallic shell (e.g., 15-25 nm thick gold).

The term “nanoparticle” as used herein refers to a particle having an average diameter of about 1-1000 nm.

The term “bio-nanoshell” as used herein refers to a nanoshell wrapped with a cell-derived biological membrane.

The term “cell-derived biological membrane” as used herein refers to a plasma membrane obtained from a cell, which comprises a phospholipid bilayer and membrane proteins from the source cell.

The term “adhesion protein” as used herein refers to a cell surface protein that binds specifically to a cognate receptor on a target cell or in the extracellular matrix.

The term “extracellular matrix” as used herein refers to a network of proteins and other molecules that surround, support, and give structure to cells and tissues.

The term “photothermal” as used herein refers to the ability of a material, for example, a bio-nanoshell, to convert light energy into heat.

The present invention provides a bio-nanoshell for binding specifically to a target cell. The bio-nanoshell comprises a cell-derived biological membrane from a donor cell. The bio-nanoshell also comprises a nanoshell. The nanoshell has an exterior surface. The exterior surface is coated with the cell-derived biological membrane. The cell-derived biological membrane comprises a phospholipid bilayer and an adhesion protein. The adhesion protein is specific for the target cell.

The bio-nanoshell may be photothermal. The bio-nanoshell may produce thermal energy when excited or activated with light of an appropriate wavelength. For example, when the photothermal bio-nanoshell is activated with light at a wavelength near the bio-nanoshell's peak plasmon resonance wavelength (also known as the wavelength of maximum absorbance or maximum extinction), the bio-nanoshell can generate heat. The activating light may be either continuous wave or pulsed.

The bio-nanoshells may have an average diameter of about 1-1000, 1-500, 1-100, 1-50, 10-1000, 10-500, 10-100, 10-50, 50-1000, 50-500, 50-100, 100-1000, 100-500, or 500-1000 nm.

The nanoshells may have an average diameter of about 50-100, 50-150, 50-200, 50-250, 50-300, 50-350, 50-400, 50-450, 50-500, 100-150, 100-200, 100-250, 100-300, 100-350, 100-400, 100-450, 100-500, 150-200, 150-250, 150-300, 150-350, 150-400, 150-450, 150-500, 200-250, 200-300, 200-350, 200-400, 200-450 or 200-500 nm. For example, the nanoshells may have an average diameter of about 100-350 nm.

The nanoshell comprises a core surrounded by a metallic shell. The dielectric core may comprise silica (also known as silicon dioxide, SiO₂), aluminum oxide, silicon oxide, titanium dioxide, polymers, gold sulfide, or a combination of these. The dielectric core may have an exterior surface. At least about 50%, 60%, 70%, 80%, 90% or 100% of the exterior surface of the dielectric core may be wrapped with the metallic shell. The dielectric core may have a diameter of about 10-100, 10-200, 10-300, 10-400, 10-500, 50-100, 50-200, 50-300, 50-400, 50-500, 100-110, 100-120, 100-130, 100-140, 100-150, 100-160, 100-170, 100-180, 100-190, 100-200, 100-300, 100-400, 100-500, 110-130, 110-140, 110-150, 110-160, 110-170, 110-180, 110-190, 110-200, 110-300, 110-400, 110-500, 120-130, 120-140, 120-150, 120-160, 120-170, 120-180, 120-190, 120-200, 120-300, 120-400, 120-500, 200-300, 200-400, 200-500, 300-400, 300-500, or 400-500 nm. For example, the dielectric core may have a diameter of about 110-130 nm.

The metallic shell may comprise gold, silver, copper, platinum, palladium, lead, or iron. Gold or silver may be preferred. The metallic shell may have a thickness of about 1-10, 1-20, 1-30, 1-40, 1-50, 10-20, 10-30, 10-40, 10-50, 15-20, 15-25, 15-30, 15-40, 15-50, 20-25, 20-30, 20-35, 20-40, 20-50, 30-40, 30-50, or 40-50 nm. For example, the metallic shell may have a thickness of about 15-25 nm. The exterior surface of the nanoshell may be the exterior surface of the metallic shell. At least about 50%, 60%, 70%, 80%, 90% or 100% of the exterior surface of the nanoshell may be wrapped with the cell-derived biological membrane. The metallic shell may be linked to the dielectric core through a linker. The linker may be aminopropyltrimethoxysilane or aminopropyltriethoxysilane or any molecule that is capable of binding both the core and the shell.

The nanoshell may have a wavelength absorbance maximum between 300 nm and 1500 nm. The bio-nanoshell may have a wavelength absorbance maximum between 300 nm and 1500 nm. The ratio of the core diameter to shell thickness of the nanoshell may be adjusted to tune the extinction profile and shift the peak plasmon resonance of the nanoshell. Nanoshells with a core diameter of about 110-130 nm and a shell thickness of about 15 to 25 nm may have a peak plasmon resonance within the near-infrared region.

The target cell may be a cell associated with the disease or disorder being treated. Where the bio-nanoshells are used to image and/or treat cancer, the target cell may be a cancer cell, cancer-associated fibroblast, tumor-associated macrophage, or other cell type present in the tumor microenvironment. The donor cell may be a cancer cell. The donor cell may be a macrophage. The donor cell may be a fibroblast. The donor cell may be a leukocyte. The donor cell may be a platelet. The donor cell may be a megakaryocyte. The donor cell may be a mesenchymal stem cell. The target cell and the donor cell may be of the same type of cells. The target cell and the donor cell may be of different types of cells.

The target cell may be in a subject. The donor cell may be from the subject. The subject may be a human or veterinary subject.

The cell-derived biological membrane is a phospholipid bilayer and one or more proteins, for example, adhesion proteins, of a donor cell. The cell-derived biological membrane lacks nuclear, cytosolic, or mitochondrial components of the donor cell. The adhesion proteins within the cell-derived biological membrane may include “markers of self” that provide immune evasion properties or proteins that enable the bio-nanoshell to bind a target cell. The biological membrane may have of thickness of about 2-50 nm. The bio-nanoshell is capable of binding specifically the target cell via the cell-derived biological membrane. The bio-nanoshell may be capable of binding an extracellular matrix via the cell-derived biological membrane.

Where the donor cell is a cancer cell, the adhesion protein may be selected from the group consisting of CD47, Na⁺/K⁺-ATPase, Thomsen-Friedenreich antigen, E-cadherin, CD44, CD326 (EpCAM, epithelial cell adhesion molecule), Pan-cadherin, Galectin-3, and VCAM-1. In the embodiment where the donor cell is a fibroblast, proteins may be selected from the group including CD47, alpha-smooth muscle actin (α-SMA), fibroblast activation protein-alpha (FAP), vimentin, and platelet-derived growth factor receptor-α (PDGFRα).

Where the donor cell is a macrophage, the adhesion protein may be selected from the group including CD47, integrins (such as α4 integrin), P-selectin glycoprotein ligand-1 (PSGL-1), L-selectin, lymphocyte function-associated antigen 1 (LFA-1), very late antigen-4 (VLA-4), Mac-1, and major histocompatibility complex (MHC) molecules.

Where the donor cell is a platelet, the adhesion protein may be selected from CD47, integrins, PECAM-1, P-selectin, CLEC-2, and GPIb.

Where the donor cells is a leukocyte, adhesion protein may be selected from CD47, CD192, VCAM-1, ICAM-1, CD45, CD11a, Mac-1, and integrins (such as α2β1, α4, β1).

Where the donor cells is a mesenchymal stem cell, the adhesion protein may be selected from CD47, CXCR4, integrins, and cell adhesion molecules (e.g., VCAM-1, ICAM-1).

For each bio-nanoshell of the present invention, a method for preparing the bio-nanoshell is provided. The preparation method comprises combining nanoshells with cell-derived biological membranes from donor cells. Each cell-derived biological membrane comprises a phospholipid bilayer and an adhesion protein specific for the target cells. The preparation method also comprises coating an exterior surface of each nanoshell with a cell-derived biological membrane.

The preparation method may further comprise extracting the cell-derived biological membranes from the donor cells so that a cell-derived biological membrane is obtained. The extraction may include steps of hypotonic lysis, homogenization, and/or multi-step centrifugation to remove intracellular components of cells and collect the resulting plasma membrane as the cell-derived biological membrane. The cell-derived biological membrane could also be produced by freeze-thaw and/or electroporation methods.

The preparation method may further comprise extruding the extracted cell-derived biological membranes through a porous extruder filter. The porous extruder filter may have a diameter of about 100-5,000 nm to form membrane vesicles. The temperature of the extruder may be set at about 37-100, 80-90, or 84-86° C. The preferred temperature may be approximately 85° C.

The cell-derived biological membrane, for example, in the form of the membrane vesicles, may be adhered or bound to the nanoshell by an electrostatic interaction. The asymmetric charge of the cell-derived biological membrane may facilitate “right-side-out” orientation on the nanoshell owing to charge repulsion between the negative extracellular membrane components and the negative surface charge of the nanoshell.

The preparation method may comprise combining the cell-derived biological membrane, for example, in the form of membrane vesicles, with the nanoshells. The mixing may be accomplished by, for example, sonication, microfluidic mixing, or co-extrusion. For co-extrusion, the porous extruder filter may have a diameter of about 100-5,000 nm to form membrane vesicles. The temperature of the extruder may be set at about 37-100, 80-90, or 84-86° C. The preferred temperature may be approximately 85° C. The number ratio of the cell-derived biological membrane, for example, in the form of membrane vesicles, to the nanoshells may be between 20:1 to 1:20. A preferred ratio may be between 10:1 to 1:1. An excess of cell-derived biological membrane vesicles to nanoshells may ensure more complete wrapping of the nanoshell exterior surface.

For each preparation method of the present invention, bio-nanoshells prepared according to the preparation method are provided.

For the bio-nanoshells of the present invention, a method for delivering the bio-nanoshells to target cells is provided. The delivery method comprises exposing the target cells to bio-nanoshells. Each of the bio-nanoshells comprises a nanoshell having an exterior surface coated with a cell-derived biological membrane from a donor cell. Each of the cell-derived biological membranes comprises a phospholipid bilayer and an adhesion protein specific for one of the target cells. The delivery method also comprises binding the bio-nanoshells specifically to the target cells via the cell-derived biological membranes. At least about 1%, 5%, 10%, 20%, 30%, 40%, 50%, 60%, 70%, 80%, 90% or 100% of the target cells may be bound specifically by the bio-nanoshells.

The target cells may be in a subject. The donor cells may be from the subject. The donor cell may be from an allogeneic source. The donor cell may be from an established cell line. The delivery method may further comprise administering the bio-nanoshells to the subject.

Where the bio-nanoshells are photothermal, the delivery method may further comprise applying light to the photothermal bio-nanoshells. The delivery method may further comprise emitting heat by the photothermal bio-nanoshells upon light application. The delivery method may further comprise killing the target cells by the heat emitted by the photothermal bio-nanoshells upon light application. At least about 10%, 20%, 30%, 40%, 50%, 60%, 70%, 80%, 90% or 100% of the target cells may be killed.

The delivery method may further comprise altering a function of the target cells by the heat emitted by the photothermal bio-nanoshells upon light application. The heat may lead to protein denaturation, DNA damage, genetic alterations, heat shock protein production, membrane damage, organelle damage, impaired mitochondrial function, or cell cycle arrest in the target cells. The function of the target cells may be selected from the group consisting of membrane permeability and fluidity, cell viability, cell motility, cell migration, cell invasion, protein synthesis, cell cycle progression, metabolic processes, and inflammatory responses. At least about 10%, 20%, 30%, 40%, 50%, 60%, 70%, 80%, 90% or 100% of the target cells may have altered function.

Where the target cells are in a subject suffering from a disease or condition, the delivery method may further comprise treating the disease or condition with the heat emitted by the photothermal bio-nanoshells upon light application.

The delivery method may further comprise emitting photoluminescence or scattering light by the photothermal bio-nanoshells upon light application.

Where the target cells are in a biological tissue, the delivery method may further comprise applying light to the photothermal bio-nanoshells, and emitting photoluminescence or scattering light by the photothermal bio-nanoshells. The delivery method may further comprise detecting the emitted photoluminescence or scattered light, wherein the presence of the emitted photoluminescence or scattered light indicates the presence of the target cells. The photoluminescence may be detected using a multiphoton microscope. The scattered light may be detected using a darkfield microscope or a reflectance confocal microscope. The image contrast of the target cells may be enhanced by the emitted photoluminescence or scattered light by at least 5%, 10%, 20%, 30%, 40%, 50%, 60%, 70%, 80%, 90%, 100%, or 200%.

Where the target cells are in a biological tissue, the delivery method may further comprise applying light to the photothermal bio-nanoshells. The photothermal bio-nanoshells may emit heat. The biological tissue may thermoelastically expand. An acoustic wave may be generated due to the thermoelastic tissue expansion.

Where the target cells are in a biological tissue, the delivery method may further comprise applying light to the photothermal bio-nanoshells and generating an acoustic wave by the biological tissue. The delivery method may further comprise detecting the acoustic wave, wherein the presence of the acoustic wave indicates the presence of the target cells.

The acoustic wave may be detected using an ultrasound transducer of a photoacoustic imaging system. The image contrast of the target cells may be enhanced by the acoustic wave by at least 5%, 10%, 20%, 30%, 40%, 50%, 60%, 70%, 80%, 90%, 100%, or 200%.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### Example 1. Synthesis of Cell Membrane-Coated Nanoshells and their Application as Photothermal Agents

In one embodiment of this invention, the inventors coated nanoshells with biological membranes derived from murine 4T1 triple-negative breast cancer (TNBC) cells and demonstrated the use of these membrane-wrapped nanoshells (i.e., MWNS or BioNS) to target TNBC cells in vitro and in vivo for enhanced photothermal therapy and photoacoustic imaging. Details of this work are provided below. Figures typically use MWNS to refer to BioNS, though the terms are used interchangeably herein.

First, nanoshells (NS) consisting of ˜120 nm diameter silica cores surrounded by ˜15-20 nm thick gold shells were synthesized according to the Oldenburg method (Oldenburg et al. Chemical Physics Letters, 1998; 288: 243-247). Briefly, 3-5 nm diameter gold colloid was made by the Duff method (Duff and Baiker, Langmuir, 1993; 9: 2301-2309) using components of hydrogen tetrachloroaurate (III) hydrate (HAuCl₄), tetrakis(hydroxymethyl)phosphonium chloride, and 1 N sodium hydroxide. The gold colloid was combined with 120 nm silica nanospheres functionalized with 3-aminopropyltriethoxysilane (purchased from Nanocomposix) and 1 M sodium chloride and rocked for 3-7 days at room temperature to create “seed” particles. The seed particles were purified twice via centrifugation and resuspended in Milli-Q water to an optical density (OD) of 0.1 at 530 nm as measured using a Cary60 spectrophotometer. Different ratios of the diluted seed were mixed with HAuCl₄ diluted in potassium carbonate and 37% formaldehyde to determine the combination that would produce NS with maximum extinction near 808 nm; this ratio was scaled up to produce the desired volume of NS. Synthesized NS were stored in water at ˜6×10⁹ NS/mL (OD^{808 nm}=2) at 4° C. While the inventors synthesized nanoshells in-house, nanoshells are also available commercially (e.g., from Nanocomposix).

4T1 and 4T1-Luc2 murine mammary breast cancer cells were purchased from American Type Culture Collection (ATCC) and cultured in Roswell Park Memorial Institute-1640 (RPMI-1640) medium supplemented with 10% fetal bovine serum (FBS) and 1% penicillin-streptomycin. EpH4-Ev murine non-cancerous breast epithelial cells (ATCC) were cultured in Dulbecco's Modified Eagle's Medium (DMEM) supplemented with 10% bovine calf serum (BCS) and 1.2 g/mL puromycin dihydrochloride 99%. Cells were maintained at 37° C. in a 5% CO₂ humidified environment and passaged or seeded for experiments when they reached 80-90% confluency by detaching the cells from the flask using 0.25% trypsin-EDTA.

4T1 or 4T1-Luc2 cell membrane vesicles were harvested from cells as follows. Cells were grown in 300 cm² cell culture flasks to 80-90% confluency and detached by adding 8 mL of 0.25% trypsin-EDTA for 3-5 min. Next, 12 mL of complete RPMI-1640 media was added to neutralize the trypsin and the cell suspension was centrifuged at 300 rcf for 7 min to obtain a cell pellet. After pelleting, cells were washed 3 times by dispersing them in 10 mL of ice cold 1× phosphate-buffered saline (PBS) and centrifuging at 300 rcf for 8 min. After washing, the pellet was suspended in 1 mL of hypotonic lysis buffer (consisting of 20 mM UltraPure 1 M Tris-HCl Buffer (pH 7.5), 10 mM KCl, 2 mM MgCl₂) and 10 μL of Halt Protease Inhibitor Cocktail, EDTA-free (100×) and left on ice for 15 min. The cell suspension was transferred to a Dounce Tissue Grinder (Kimble) and emulsified by doing 30 passes with 2 different size pestles (A & B). The emulsified cell suspension was then added to two cold 1.5 mL Eppendorf tubes and centrifuged at 1000 rcf for 10 min at 4° C. The supernatant from each tube was transferred to a new ice cold 1.5 mL Eppendorf tube. Remaining cell pellets were then broken up by adding 500 μL of hypotonic lysis buffer and 5 μL of protease inhibitor to each pellet. Cell suspensions were then combined and added into the Dounce Tissue Grinder, emulsified with both pestles, and centrifuged in two balanced, separate 1.5 mL tubes at 1000 rcf or 10 min at 4° C. The supernatants from both tubes were then combined into a new cold 1.5 mL tube, and the pellets (containing nuclear components) were discarded. The supernatants collected from the previous steps were then centrifuged at 10,000 ref for 10 min at 4° C. and the pellets (containing mitochondrial components) were discarded. The supernatants were then placed in 1.5 mL polypropylene tubes and centrifuged at 40,000 rpm for 90 min at 4° C. (Beckman Optima MAX Benchtop Ultracentrifuge). The supernatants (containing cytosolic components) were discarded, and the pellet (cell membrane fragments) was resuspended in 1200 μL Milli-Q water. The resulting membrane fragments were purified by physical extrusion through an 0.4 m polycarbonate membrane (Avanti Polar Lipids) in a mini-extruder (Avanti Polar Lipids) at 85° C. for 11 passes to produce monodisperse cancer cell membrane vesicles (cell MVs). Note that the 85° C. utilized here is higher than typically reported extrusion temperatures (which range from ˜37° C. to ˜45° C.).

The hydrodynamic diameter and zeta potential of the bare NS (OD^{800nm}=1) and cell MV samples diluted in Milli-Q water were measured using a LiteSizer 500 (AntonPaar) dynamic light scattering (DLS) instrument. Each sample's concentration (particles/mL) was measured using a NanoSight NS300 nanoparticle tracking analysis system (NTA; Malvern Analytical). To visualize the morphology of the samples, electron micrographs were acquired using a Talos F200C Transmission Electron Microscope (TEM). TEM samples were prepared by plasma-cleaning carbon support films on 300-mesh copper grids for 30 seconds, followed by the addition of bare NS or cell MV samples for 30 seconds. The grids were then washed four times with deionized (DI) water, stained with 2% uranyl acetate for 15 seconds, air-dried, and imaged.

Using the concentration (particles/mL) measurements recorded from the NTA, the cell MV and bare NS were combined at a 4:1 membrane/NS ratio to produce membrane-coated NS (i.e., MWNS, also known as BioNS). This solution was bath sonicated for 30 see then extruded through a 0.4 m polycarbonate membrane (Avanti Polar Lipids) in a mini-extruder (Avanti Polar Lipids) at 85° C. for 11 passes to produce 4T1 cancer cell membrane-coated gold NS (MWNS). The final MWNS solution was collected in a 1.5 mL Eppendorf tube and centrifuged at 500 rcf for 10 min at 23° C. to pellet the MWNS and remove any unused membranes in solution. The centrifuged MWNS pellet was resuspended in Milli-Q water and used immediately or stored at 4° C. The MWNS hydrodynamic diameter and zeta potential were measured using the LiteSizer instrument and their concentration (particles/mL) was measured using the NanoSight NS300 NTA. To visualize the morphology of MWNS, TEM images were acquired using a Talos F200C TEM. TEM samples were prepared by plasma-cleaning carbon support films on 300-mesh copper grids for 30 seconds, followed by the addition of MWNS samples for 30 seconds. The grids were then washed four times with DI water, stained with 2% uranyl acetate for 15 seconds, air-dried, and imaged.

As a control, PEG-coated NS (PEG-NS) were synthesized by adding 5 kDa methoxy-poly(ethylene glycol)-thiol (m-PEG-SH; Laysan Bio) diluted in Milli-Q water to 1 mM to bare NS at a final concentration of 20 μM. After rocking overnight at 4° C., the solution was purified twice via centrifugation to remove unbound mPEG-SH. The purified PEG-NS were stored at 4° C.

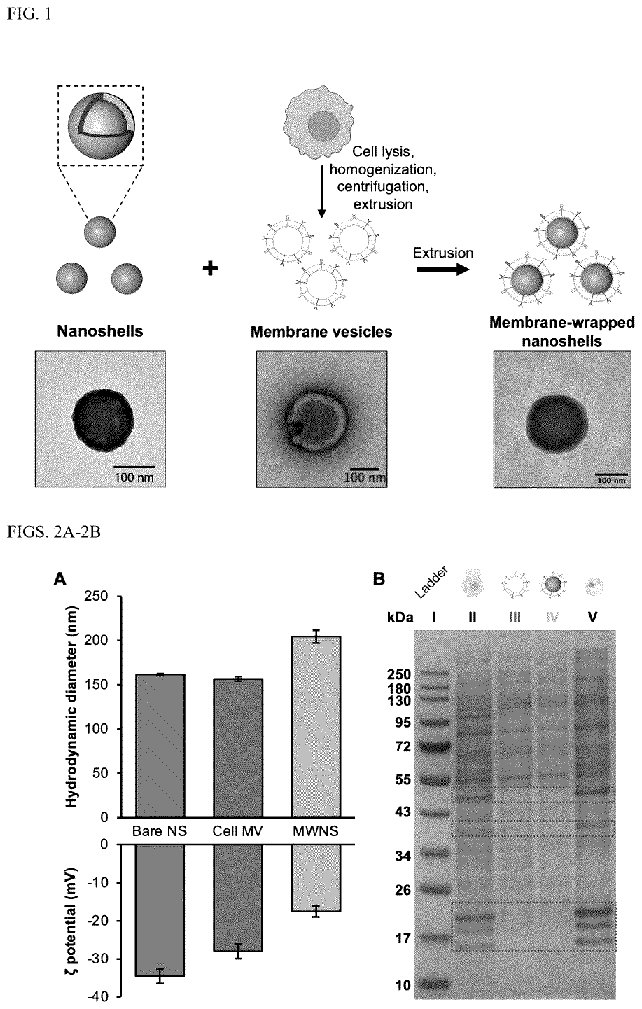

FIG. 1 shows a simplified scheme of the synthesis process along with TEM images of bare NS, membrane vesicles extracted from 4T1 cells, and the final 4T1 membrane-wrapped NS (i.e., MWNS or BioNS). The NS are spherical in structure and a membrane coating is visible around the NS core in the MWNS sample image. It was surprising that membranes could be successfully extracted and coated upon NS using a temperature of 85° C. during the extrusion processes, as this temperature is much higher than the temperatures reported in prior works describing the production of membrane-wrapped nanoparticles (which ranged from ˜37 to ˜45° C.). Many proteins and lipids may denature at temperatures above 45° C., but the precise temperature of denaturation depends on the specific proteins or lipids, the length of time the heat is applied, and factors in the surrounding environment such as pH, salt concentration, and presence of other molecules. The inventors found that a temperature of 85° C. could be used during extrusion to wrap membranes around NS and, as discussed below, the proteins in the membranes maintained the ability to bind target cells once upon the NS surface. While the membrane extraction and NS coating could be performed at a lower temperature, the inventors found the higher temperature of 85° C. allowed for improved flow and BioNS production due to reduced viscosity.

The hydrodynamic diameter and zeta potential of bare NS, extracted 4T1 cell MVs, and 4T1 membrane-wrapped NS (MWNS/BioNS) are shown in FIG. 2A. Compared to bare NS, the MWNS have a larger hydrodynamic diameter and more neutral zeta potential, indicative of successful surface modification. The hydrodynamic diameter and zeta potential of the Bare NS and MWNS samples were also measured after being placed in water at 4° C. for 10 days (to mimic storage conditions) or in pH7.4 PBS containing 10% FBS at 37° C. for 24 hours (to mimic physiological conditions). The hydrodynamic diameter and zeta potential of the MWNS remained unchanged after incubation in storage of physiologic conditions, demonstrating their stability.

During the membrane extraction protocol, samples of the 4T1 whole cell lysate, cell nuclear components, and mitochondrial components were kept and stored on ice. To assess protein content in BioNS and verify that the membrane coating consisted of only extracellular components and lacked intracellular components, sodium dodecyl sulfate polyacrylamide gel electrophoresis (SDS-PAGE) was performed (FIG. 2B). For this assay, a NanoDrop One instrument (Thermofisher) was used to measure the protein concentration in the 4T1 cell nuclear components, mitochondrial components, whole cell lysate, cell MVs, and BioNS (also known as MWNS). When determining protein concentration on BioNS, the background signal measured for Bare NS was subtracted from the BioNS result. Based on the protein concentrations measured in the NanoDrop One instrument, all samples were diluted to 30 g of protein with 1×PBS and 4× Bolt LDS Sample Buffer (Thermofisher) and denatured for 10 min at 70° C. while vortexing at 400 rpm. For BioNS, after protein denaturation, the sample was centrifuged at 10,000 rcf for 10 min at 23° C. to pellet bare NS. The pellet (bare NS) was discarded and the supernatant (proteins from the cell membrane layer on the surface of the MWNS) was used for the gel. SDS-PAGE was run using 30 g of protein sample per well (total volume of 30 μL per well) in 4-12% Bolt Bis-Tris Plus gels, with one well containing a pre-stained 10-250 kDa protein ladder (Cell Signaling Technologies). A mini-gel tank (Thermofisher) was filled with 1×3-(N-morpholino) propanesulfonic acid (MOPS) running buffer, and the gels were run at 120 V for 70 min. The gel was then removed from the cassette, rinsed 3× with reverse osmosis purified (RO) water, and stained with SimplyBlue SafeStain for 1 h at room temperature while rocking. The excess stain was discarded, and the gel was rinsed in RO water for 30 min while rocking at room temperature. The RO water was then discarded, and new RO water was added to destain the gel overnight while rocking at 4° C. After rinsing overnight, the water was discarded, and the gel was rinsed twice at room temperature for 2 thirty-minute intervals. Finally, the gel was placed on transparency paper and imaged for qualitative analysis. As shown in FIG. 2B, the lanes containing 4T1 cell MVs and 4T1 membrane-coated BioNS have similar band profiles. Additionally, proteins present in the whole cell lysate and nuclear pellet lanes (outlined in dotted boxes) are not present in the MV or BioNS lanes, confirming these nuclear proteins have been removed during the membrane extraction process.

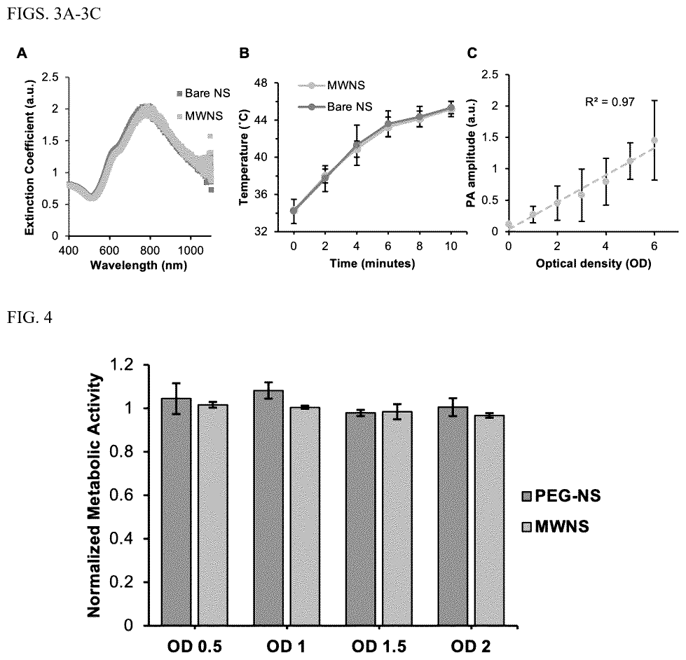

The inventors next characterized the optical properties of BioNS as compared to Bare NS. BioNS or Bare NS samples (diluted in Milli-Q water) were placed in 1-cm pathlength disposable cuvettes and analyzed on a Cary 60 UV-vis spectrophotometer (Agilent) from 1100 nm to 400 nm to obtain their extinction spectrum. As shown in FIG. 3A, the Bare NS and BioNS both have peak extinction near 800 nm, indicating the membrane coating does not alter the optical properties of the NS. The photothermal performance of Bare NS and BioNS placed in an aqueous solution (i.e., their ability to convert light to heat) was evaluated under continuous wave laser irradiation. A solution of 500 μL BioNS or Bare NS at 10¹⁰ NS/mL (corresponding to an optical density of 5 at the peak extinction wavelength (OD⁸⁰⁸=5)) in a disposable cuvette with a magnetic stir bar was heated on a stir plate set at 300 rpm and 45° C. for 1.5 h prior to irradiation to bring the starting temperature to ˜34° C. The solution was then irradiated with an 808 nm continuous wave BWF-2 series laser (B&W TEK Inc) at 1.5 W/cm² for 10 min while monitoring the maximum temperature in each sample with an FLIR 6EX series thermal camera (FLIR) every 2 minutes. Bare NS and BioNS exhibited the same level of heating upon irradiation (FIG. 3B), indicating that the membrane coating does not hinder the photothermal conversion abilities of the nanoparticles. The inventors also characterized the photoacoustic (PA) signal that could be generated by BioNS (FIG. 3C). For PA signal characterization, BioNS samples at different optical densities (OD⁸⁰⁸=0, 1, 2, 3, 4, 5, and 6) were prepared with the same solvent (Milli-Q water) and ˜60 μL were injected into polyethylene tubes (FujiFilm VisualSonics Inc) that were placed horizontally in a block. The block was then filled with water until the tubes were completely submerged. For PA imaging, the tubes were lased in the spectral range from 970 to 680 nm with 2 nm intervals using a UHF46x high frequency transducer with a Vevo F2 LAZR-X system (FujiFilm VisualSonics Inc.). Following imaging, regions of interest were drawn over the samples, and the PA signal amplitude was quantified. FIG. 3C shows that the amplitude measured at 680 nm is linearly correlated with BioNS concentration. Taken together, these experiment reveal that BioNS maintain the inherent optical and photothermal properties of unwrapped NS that can enable their use as contrast agents or photothermal therapeutics.

To confirm BioNS are biocompatible in the absence of irradiation/light exposure, they were applied to cells and then metabolic activity was measured. More specifically, 4T1 cells were seeded in 96-well plates at a density of 15,000 cells per well and incubated at 37° C. in a 5% CO₂ humidified incubator overnight to allow cell attachment. Then, the culture medium in the corresponding wells was replaced with fresh medium containing BioNS or PEG-NS at different concentrations (OD^{800 nm}=0, 0.5, 1, 1.5, 2, and 2.5) and samples were incubated for 4 h. Subsequently, the medium in each well was replaced with a mixture of Alamar Blue Reagent and complete culture medium at a 1:10 dilution. After incubating at 37° C. for 4 h, the Alamar Blue fluorescence was read at 560 nm excitation/590 nm emission on a Synergy H1M plate reader (BioTek). It was observed that BioNS and PEG-NS were biocompatible across all concentrations tested (FIG. 4), as there was no reduction in metabolic activity compared to untreated cells.

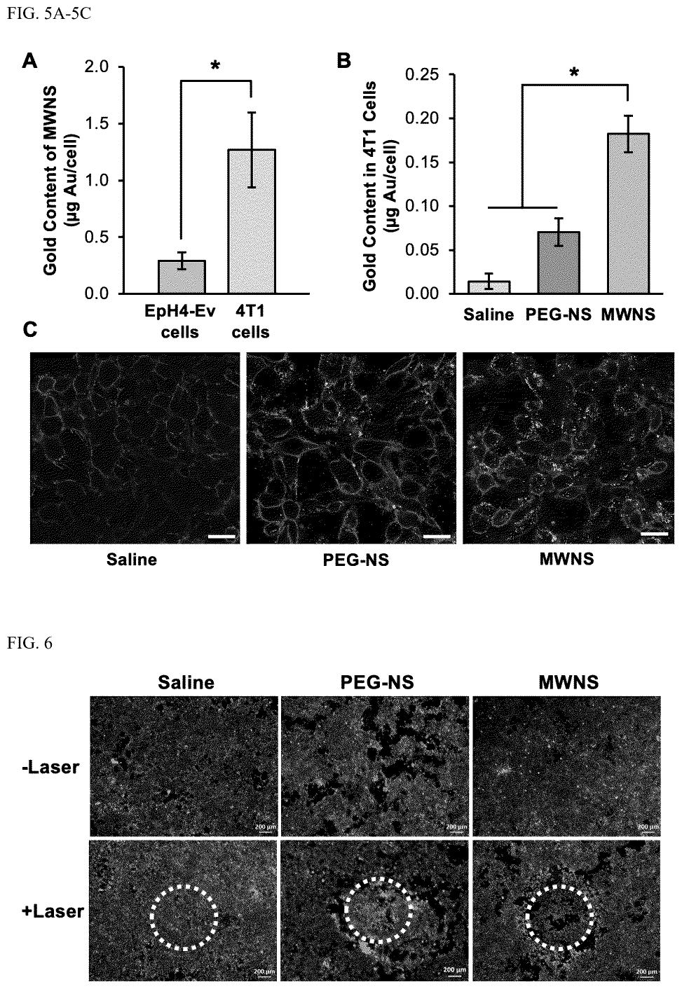

To evaluate the preferential binding of BioNS to 4T1 cells versus EpH4-Ev cells, 500,000 cells of each cell type were seeded in separate wells of a 6 well plate and incubated at 37° C. in a 5% CO₂ humidified incubator overnight for cell attachment. The cells were then treated with 2 mL of BioNS at OD⁸⁰⁸=1 in serum-containing medium and incubated for 4 h at 37° C. Cells were then rinsed twice with warm PBS containing calcium and magnesium (Invitrogen) by thorough gentle pipetting around the well to remove any unbound/non-internalized particles. Cells were then trypsinized, neutralized with 1×PBS, and the cell suspension was centrifuged at 300 rcf for 5 min to obtain a cell pellet. The supernatant was discarded, and the pellet was resuspended 1×PBS and counted with a hemocytometer. An aqua regia solution (consisting of 97% trace metal grade hydrochloric acid and 3% trace metal grade nitric acid) was added to the cell suspension in a 1:1 ratio and placed in a heat block at 60° C. in a fume hood for 30 min. Next digested samples were diluted 1000-fold in an acid matrix containing 2% nitric acid and 2% hydrochloric acid in MilliQ water, and a standard curve containing 0-100 parts per billion (ppb) gold using TraceCert Gold Standard for ICP (Sigma) was prepared. An internal baseline of 5 parts per million (ppm) was added to each standard and sample. Samples were analyzed on a 7500C inductively coupled plasma-mass spectrometer (ICP-MS, Agilent) and the gold content in ppb was recorded for each sample. The concentration of gold determined by ICP-MS was converted to μg Au and reported as μg Au per cell. The BioNS exhibited significantly greater uptake by 4T1 cells than by EpH4-Ev cells (FIG. 5A), demonstrating the membrane coating enables preferential targeting of homotypic cells.

The interaction of BioNS versus PEG-NS with 4T1 cells was assessed via ICP-MS following the same method described above. FIG. 5B shows that BioNS exhibited greater binding to/uptake by 4T1 cells than PEG-NS. Multiphoton microscopy was also used to qualitatively visualize nanoparticle binding to/uptake by 4T1 cells. For this, 4T1 cells were seeded in an 8-well Nunc Lab-Tek™ Chambered Coverglass, Borosilicate Glass 1.0 slide (Thermofisher) at 40,000 cells per well and left at 37° C. in a 5% CO₂ humidified incubator to adhere overnight. Cells were then treated with 300 μL of BioNS or PEG-NS at OD⁸⁰⁸=1 in serum-containing medium or left untreated and incubated for 6 h at 37° C. After 6 h, the cells were gently rinsed 3× with warm DPBS to remove unbound particles. Cells were then fixed with 4% formaldehyde at room temperature for 15 min and neutralized with 1×PBS afterwards. Cell membranes were dyed using CellVue Claret Red Membrane Dye for 10 minutes at room temperature, according to the manufacturer's protocol. The dye was neutralized with 1% w/v Bovine Serum Albumin (BSA) in 1×PBS. The dye solution and neutralizer were then removed, and the cells were rinsed with 1×PBS. A final volume of 200 μL of 1×PBS was added to each well prior to imaging with a LSM880 Multiphoton Confocal Microscope (Zeiss) with a 20×/0.8 water objective, which was immersed in a droplet of water to provide the correct contrast and visualization upon connection to the slides. The NS in the samples were excited by the multiphoton laser tuned to λ_{excitation}=800 nm with a pinhole of 1.57 AU and a detection range λₑₘᵢₛₛᵢₒₙ of 400-550 nm. The cell membranes in the slide samples were visualized at λ_{excitation}=655 nm and λₑₘᵢₛₛᵢₒₙ of 675 nm. FIG. 5C shows 4T1 cells bound and/or internalized more BioNS than PEG-NS. Collectively, the ICP-MS and multiphoton microscopy data reveal that BioNS are preferentially taken up by homotypic 4T1 cells and that the 4T1 cells internalize/bind more BioNS than PEG-NS.

The ability of BioNS to mediate photothermal therapy and serve as photoacoustic imaging contrast agents was next explored in vitro. For photothermal therapy studies, 4T1 cells were plated in a 24-well plate at a density of 80,000 cells per well and incubated at 37° C. in a 5% CO₂ humidified incubator overnight for proper cell attachment. Cells were incubated with complete media supplemented with BioNS or PEG-NS at OD⁸⁰⁸=1.5 or left untreated for 4 h. After 4 h, the cells were gently rinsed 3× with warm DPBS to remove unbound particles and replenished with fresh media. Cells were then exposed to an 808 nm continuous wave BWF-2 series laser (B&W TEK Inc) with a 2 mm diameter spot size at 60 W/cm² for 2 minutes and then incubated for 1.5 h at 37° C. A Viability/Cytotoxicity Assay Kit (Biotium) that contains calcein AM and ethidium homodimer III (EthD-III) was used to assess cell membrane integrity. In live cells, the nonfluorescent calcein AM molecule is converted to green, fluorescent calcein after hydrolysis by intracellular esterases, resulting in green fluorescence. In contrast, EthD-III remains weakly fluorescent until it binds to DNA, emitting strong red fluorescence upon binding. Since EthD-III is impermeable to cells with an intact plasma membrane, its uptake serves as an indicator of loss of membrane integrity/cell death. In this assay, a staining solution consisting of 2 μM calcein AM and 4 M EthD-III diluted in 1×PBS was added to the cells and incubated for 20 min at room temperature. The cells were then rinsed with 1×PBS. A final volume of 500 μL of 1×PBS was added to each well prior to imaging with an AxioObserver Z1 microscope (Zeiss). In the absence of laser irradiation, BioNS and PEG-NS did not hinder cell viability (FIG. 6, top row). Upon irradiation, both PEG-NS and BioNS were able to induce photothermal cell death (FIG. 6, bottom row; irradiated regions are within the circled area), but BioNS were more effective than PEG-NS, as evidenced by the more complete loss of cell viability within the irradiated region.

To evaluate the PA imaging capabilities of BioNS, a tissue-mimicking phantom base comprising 8% m/v gelatin (Sigma), 0.2% m/v silica (Sigma), and 0.1% m/v formaldehyde was prepared according to a protocol modified from literature (Cook et al., Biomedical Optics Express, 2011; 2(11): 3193-3206). Briefly, a mixture consisting of 300 mg of 0.4 m diameter silica particles and 150 mL Milli-Q water in a beaker with a magnetic stir bar was heated on a stir plate set at 700 rpm and 65° C. for 2.5 h while covered to minimize vapor loss. To enhance crosslinking and improve gel stability, a 150 mg (˜4 mL) of 37% formaldehyde was added and stirred for 3 min at 45° C. before degassing the solution in a vacuum chamber for 10 min. Very slowly, 12 g of type-A, 300-Bloom gelatin powder derived from acid-cured porcine skin was added and stirred at 200 rpm for 10 min at 45° C. A metal scraper was used to gently remove air bubbles on the surface. The solution was then poured in a silicon mold and allowed to cure for 20 min at room temperature, then placed into a 4° C. refrigerator overnight to fully cross-link. In this phantom base, gelatin served as the primary matrix, and the silica particles were added to mimic the acoustic scattering properties of soft tissues.

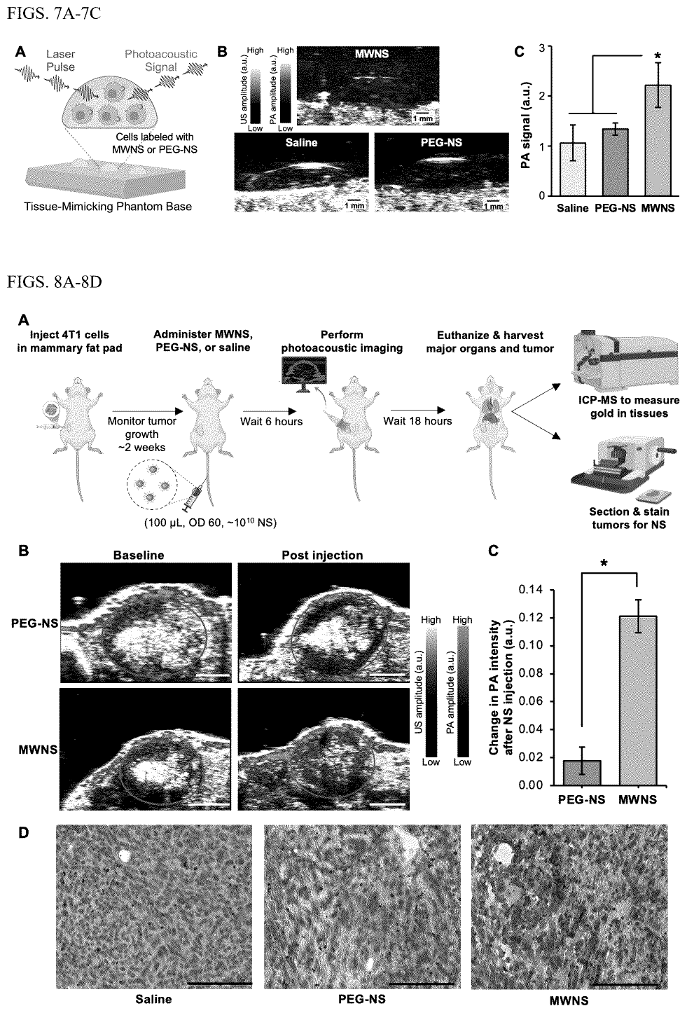

Separately, 4T1 cells were plated in a 6-well plate at a density of 150,000 cells per well and incubated at 37° C. in a 5% CO₂ humidified incubator for 48 h. Cells were incubated with complete media supplemented with BioNS or PEG-NS at OD⁸⁰⁸=0.5 (1.4×10⁹ NS/mL) or left untreated for 6 h. After 6 h, the cells were gently rinsed 2× with warm 1×PBS to remove unbound particles. Cells were then trypsinized, neutralized with 1×PBS without calcium and magnesium, and the cell suspension was centrifuged at 300 rcf for 5 min to obtain a cell pellet. The supernatant was discarded, and the pellet was resuspended 1×PBS and counted with a hemocytometer. Cells were the dispersed in PBS with the final concentration of 1200 cells per L. For the gelatin-based dome phantom filled with cells, a 16% w/v gelatin solution was prepared and heated up to 80° C. Next, the heated gelatin solution was mixed with the cells in a volume ratio of 1:1. The cell-gelatin mixture was then pipetted onto the phantom base to establish dome-shaped droplets, which were stored at 4° C. overnight to fully crosslink and then imaged using a Vevo F2 LAZR-X system (FujiFilm VisualSonics Inc.) (FIG. 7A). PA images of the cell droplets show minimal signal in saline-treated cells, intermediate signal for PEG-NS-treated cells, and the highest signal for BioNS-treated cells (FIG. 7B). Quantitative analysis revealed that the PA signal in BioNS-treated cells was significantly higher than that in PEG-NS or saline treated samples (FIG. 7C).

The inventors next investigated the ability of BioNS to accumulate in tumors in vivo to enable photoacoustic imaging of tumors as well as photothermal tumor ablation. All in vivo experiments were performed under animal use protocol (AUP) 1400 approved by the University of Delaware Institutional Animal Care and Use Committee (IACUC). Female immune competent Balb/cJ mice around 6-8 weeks old were purchased from Jackson Laboratory. Upon arrival to the animal facility, mice were maintained on a low chlorophyll diet to minimize autofluorescence and left to acclimate for 1 week prior to tumor inoculation surgery. Before tumor inoculation surgery, luciferase-expressing 4T1-Luc2 cells (60-70% confluency) were trypsinized, collected in complete-culture medium, and pelleted at 200 rcf for 5 min. The supernatant was aspirated, and the pellet was washed twice in 1×PBS without magnesium and calcium via centrifugation. Cells were counted and diluted in PBS at a concentration of 100,000 cell per 50 μL and kept on ice. During surgery, mice were weighed, anesthetized using 2-3% isoflurane in oxygen, and received intraperitoneal injection of analgesic buprenorphine at 0.1 mg/kg. Successful sedation was confirmed by lack of response to a toe pinch. A longitudinal incision was made between the 4^{th} nipple and the midline using a scalpel blade (MidWestVetSupply), then a cotton swab soaked with sterile PBS was inserted in the incision to create a pocket. A curved tweezer (Roboz surgical instrument) was then used to lift the skin, and a blunt tweezer (Roboz surgical instrument) was used to pull out the fat pad area. The cell suspension was slowly injected in the fat pad using a 29G 1 mL insulin syringe (MedLabSupply) and the fat pad was slowly released after inoculation. Finally, a curved tweezer was used to bring the skin at the incision together and VetBond surgical glue was applied to close the incision site. Mice were then monitored in a sterile warm cage until awake and mobile, then returned to their home cage. Successful inoculation was confirmed ˜one week after surgery using IVIS Lumina III Imaging System (PerkinElmer). For IVIS imaging, mice were weighed, anesthetized with 2-3% isoflurane in oxygen, and injected intraperitoneally with D-luciferin (Biotium) at a 150 mg/kg body weight 5 min prior to imaging. Tumor growth was monitored at least 2 times per week with digital Vernier calipers. Each mouse's weight was measured at least 2 times per week following cell implantation. When the tumor volume reached ˜100-300 mm³, mice received 100 μL of saline, BioNS, or PEG-NS intravenously through the tail vein (the BioNS and PEG-NS were at a concentration of OD60, such that ˜10¹⁰ NS were injected). Mice were then used for either a biodistribution and photoacoustic imaging study or a phototherapeutic efficacy study, detailed below.

The study design for the biodistribution/imaging study is shown in FIG. 8A. Tumors in the mice were imaged with the Vevo F2 LAZR-X system prior to NS injection to obtain baseline ultrasound/photoacoustic (US/PA) images. For US/PA imaging, mice were anesthetized using 2-3% isoflurane in oxygen and placed on a heated translation motor stage in the Vevo F2 LAZR-X imaging system. US/PA images were acquired with UHF46x high frequency transducer within the 680-920 nm spectral range in 2 nm increments. Following baseline imaging, mice received 10¹⁰ (OD⁸⁰⁸=60, 100 μL) BioNS, PEG-NS or saline intravenously, through the tail vein. Six hours post injection, US/PA images of the tumor region were acquired as described above. For each mouse, baseline image settings were saved as a preset that was loaded for the post-injection imaging session. After imaging, regions of interest were drawn inside the tumor, and the PA signal amplitude from NS was quantified using the spectral unmixing function to exclude signal contributions from oxygenated and deoxygenated hemoglobin in vivo. For each mouse the change is NS PA signal intensity was calculated by subtracting the baseline signal from the post-injection signal. FIG. 8B shows representative baseline and post-injection PA/US images for mice injected with PEG-NS or BioNS. The BioNS provide substantially higher photoacoustic signal throughout the tumor volume, which was confirmed through quantitative measures (FIG. 8C).

The day after PA imaging was performed (˜24 h post nanoparticle injection), mice were humanely euthanized by carbon dioxide asphyxiation followed by cervical dislocation, and their major organs (heart, liver, spleen, kidneys, blood, brain, lungs) and tumors were harvested for inductively coupled plasma mass spectrometry (ICP-MS) analysis to quantify the gold content in tissues. For each tumor, one half was used for ICP-MS analysis and the other half was used for histology/silver staining. For the histological examination, half of each tumor was rinsed in 1×PBS and placed into an embedding cassette (Simport). Tumor tissues were fixed in 4% paraformaldehyde (in 1×PBS) for 72 h while rocking at 4° C., then rinsed thrice in 70% ethanol for 15 min at 4° C. while rocking. Cassettes were then stored in 70% ethanol 4° C. prior to processing. Fixed tissues were processed using an ASP6025 S Tissue Processor (Leica) and embedded with paraffin using a HistoDream Embedding Center (Milestone Medical). Tissue blocks were created using ParaPro Blue paraffin (StatLab). Embedded tissues were cut into 5 μm slices using an RM 2125 rotary microtome (Leica). Tumor sections were then stained with silver staining that nucleates on the gold to enable visualization by light microscopy. Briefly, tissue sections were deparaffinized twice in xylene (Thermofisher) for 10 min, and hydrated through a series of ethanol (100%, 90%, 70%, 50%) for 5 min and thrice Milli-Q water for 3 min. Tissue were traced with a hydrophobic PAP pen (abcam) and silver stain (Cytodiagnostics Silver Enhancer Kit) was incubated on the slides at room temperature for 10 min. The samples were rinsed thrice Milli-Q water for 5 min and counterstained with Hematoxylin (Electron Microscopy Sciences) for 3 min, then rinsed in Milli-Q water followed by 37 mM ammonium hydroxide (NH₄OH) bluing agent (Sigma) for 2 min. Finally, samples were rinsed in Milli-Q water twice and mounted with an aqua-mount mounting media (Epredia), coversliped and imaged using an EVOS M7000 imaging system. The images (FIG. 8D) show more BioNS within tumor tissues as compared to PEG-NS, in agreement with the elevated PA signal observed. ICP-MS analysis of excised tissues indicated that BioNS had ˜34% greater tumor accumulation than PEG-NS. Other tissues with accumulation of BioNS and PEG-NS included the spleen and liver. Notably, the amount of BioNS in the spleen was ˜30% lower than the amount of PEG-NS, which was a statistically significant reduction.

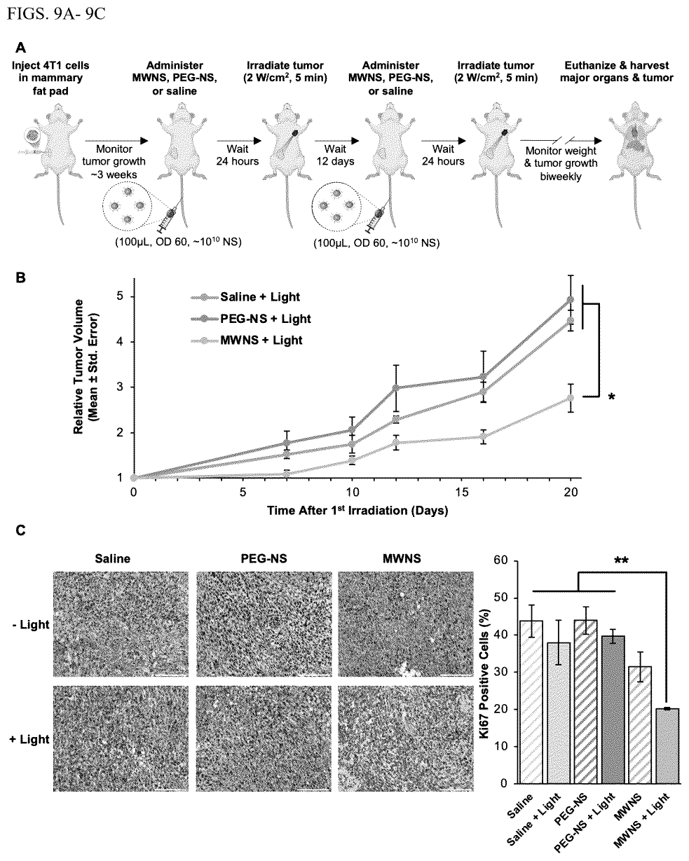

The ability of BioNS to mediate photothermal tumor ablation was also evaluated in mice bearing orthotopic 4T1 tumors as described above (scheme in FIG. 9A). When the tumor volume reached ˜100-300 mm³ (denoted as day 0), mice received 10¹⁰ (OD⁸⁰⁸=60, 100 μL) BioNS, PEG-NS or saline intravenously, through the tail vein. After 24 h, mice were anesthetized using 2-3% isoflurane in oxygen and their tumors were swabbed with glycerol as an index-matching agent prior to transdermal irradiation, with the laser diameter being set to completely cover the tumor diameter, without exposing more than ˜1 mm of the surrounding tissue. Tumors were irradiated with an 808 nm continuous wave BWF-2 series laser (B&W TEK Inc) at 2 W/cm² for 5 min. During irradiation, the tumor temperature was monitored using an FUR thermal camera. Subsequently, all six groups of mice received one additional round of injection or injection plus laser irradiation 12 days apart. Throughout the study, body weight and tumor volume were measured at least two times per week using digital Vernier calipers until the mice were euthanized. Mice were humanely euthanized when tumors reached ˜1000 mm³, when their body weight decreased by >20%, or 30 days post-irradiation, whichever came first. Mice were humanely euthanized by carbon dioxide asphyxiation followed by cervical dislocation, and the major organs (spleen, liver, lungs) and tumors were harvested for histological analysis. Excitingly, photothermal therapy mediated by BioNS slowed tumor growth significantly as compared to photothermal therapy mediated by PEG-NS or the application of light alone (FIG. 9B). Mice exposed to BioNS plus light also exhibited the least intratumoral proliferation, as demonstrated by measurement of the percentage of Ki67 positive cells in tumor histological sections (FIG. 9C). For histology, excised tumors and organs were rinsed in 1×PBS and placed into embedding cassettes (Simport). Tissues were fixed in 4% paraformaldehyde (in 1×PBS) for 72 h while rocking at 4° C., then rinsed thrice in 70% ethanol for 15 min at 4° C. while rocking. Cassettes were then stored in 70% ethanol 4° C. prior to processing. Fixed tissues were processed using an ASP6025 S Tissue Processor (Leica) and embedded with paraffin using a HistoDream Embedding Center (Milestone Medical). Tissue blocks were created using ParaPro Blue paraffin (StatLab). Embedded tissues were cut into 5 μm slices using an RM 2125 rotary microtome (Leica). Organ tissue sections were stained with hematoxylin and eosin (H&E) using the Leica Autostainer XL platform to enable visualization of tissue structure and histological changes. Briefly, all tissue sections were deparaffinized twice in xylene (Thermofisher) for 10 min, and rehydrated through a series of ethanol (100%, 90%, 70%, 50%) for 5 min and thrice Milli-Q water for 3 min prior to hematoxylin staining and subsequent counterstaining with eosin. Tumor sections were stained with antibodies against proliferating cell nuclear antigen Ki67 to evaluate cell proliferation. After staining, tissues were dehydrated and mounted with xylene-based mounting media and imaged using an EVOS M7000 imaging system.

Images of Ki67-stained tumor tissues were analyzed with QuPath image analysis software to determine the average positive cell percentage ((positively stained cells)/(total cells in image)×100) (FIG. 9C). To quantify the percentage of Ki67 positive cells in tumor images, QuPath's positive cell detection tool was utilized. Briefly, the image was loaded, and the program was trained to distinguish different cell types by using the click and drag tool to draw a selection on a certain cell type and classifying it accordingly. For example, stromal cells were excluded by classifying them as “Stroma,” and tumor cells were defined by selecting both “positive” and “negative” tumor cells then assigning them as “Tumor.” After training the classifier, a region containing different cell types was outlined using the rectangle tool, followed by Analyze >Cell Detection >Positive Cell Detection was performed using the optical density sum as the detection image. After running the detection, positive cells were highlighted in red and negative cells appeared in blue. The percentage of positive cells was highlighted in the annotation tab. For each tumor sample, seven images of distinctive cell regions were analyzed. Each sample image value was then averaged against other samples from the corresponding tumor. Each tumor value was then averaged with other tumor values from the corresponding treatment groups and analyzed using one-way ANOVA.

Notably, the 4T1 tumor model utilized in this research is able to spontaneously metastasize from the mammary fat pad to the lungs. Accordingly, lungs excised from mice were analyzed to determine any treatment effects on lung metastasis. For this, H&E-stained lung tissues were analyzed with ImageJ software to determine the average metastasis area percentage ((metastasis area)/(total tissue area) ×100). The images of lungs were downloaded into ImageJ, converted into 8-bit, and made Binary. The Wand tool was used to trace and measure total tissue area and the Freehand selection tool was used to outline and measure the areas of metastatic regions. Each metastatic region's area was then totaled and divided by total lung tissue area to determine the metastasis area percentage per sample. The combination of BioNS plus laser irradiation (i.e., photothermal therapy mediated by BioNS) reduced lung metastasis burden by ˜72% compared to the saline control (4% metastatic area versus 14.5% metastatic area), while PEG-NS plus laser irradiation (i.e., photothermal therapy mediated by PEG-NS) reduced metastasis burden by only ˜36% (9.3% metastatic area) versus the saline control. The metastatic area of the lungs in the PEG-NS and MWNS groups without light exposure was 12.4% and 11.9%, respectively, indicating the particles alone have minimal impact on metastasis.

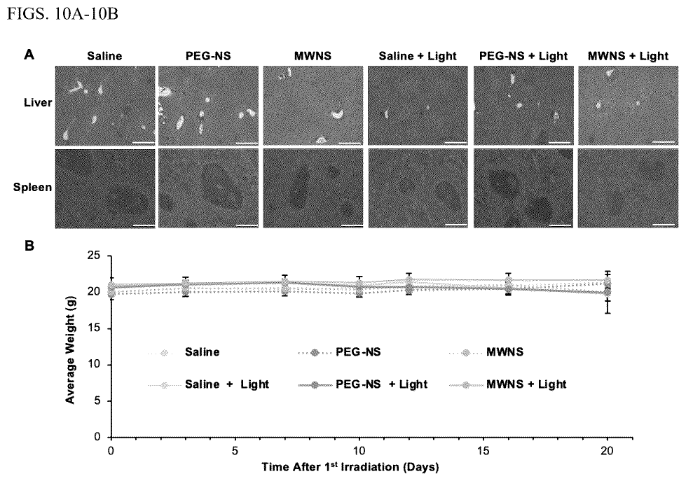

Lastly, to gain insight into the biocompatibility of BioNS in vivo, H&E-stained sections of livers and spleen were imaged. As shown in FIG. 10A, there was no obvious difference in the structure of these tissues in mice treated with BioNS or PEG-NS as compared to those treated with saline. There were also no significant differences in animal weight versus time (FIG. 10B) in mice treated with saline, BioNS or PEG-NS, providing further evidence that the nanoparticles are well tolerated.

In total, these studies demonstrate that nanoshells can be coated with cell-derived biological membranes to produce BioNS that have optical properties suitable for use as photoacoustic imaging agents and/or as photothermal therapy agents. The BioNS exhibit greater accumulation in targeted cells and tumors compared to PEG-NS, allowing for improved contrast and phototherapeutic effects. BioNS are also well tolerated in vitro and in vivo. Overall, BioNS are promising tools for targeting imaging and/or treatment of disease.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed:

1. A bio-nanoshell for binding specifically to a target cell, comprising a cell-derived biological membrane from a donor cell and a nanoshell having an exterior surface coated with the cell-derived biological membrane, wherein the cell-derived biological membrane comprises a phospholipid bilayer and an adhesion protein specific for the target cell.

2. The bio-nanoshell of claim 1, wherein the bio-nanoshell is photothermal.

3. The bio-nanoshell of claim 1, wherein the target cell is a cancer cell.

4. A method for preparing bio-nanoshells capable of binding specifically to target cells, comprising:
(a) combining nanoshells with cell-derived biological membranes from donor cells, wherein each cell-derived biological membrane comprises a phospholipid bilayer and an adhesion protein specific for the target cells; and
(b) coating an exterior surface of each of the nanoshells with one of the cell-derived biological membranes, whereby bio-nanoshells are prepared.

5. The method of claim 4, further comprising extracting the cell-derived biological membranes from the donor cells, whereby extracted cell-derived biological membranes are generated.

6. The method of claim 5, further comprising extruding the extracted cell-derived biological membranes through a porous extruder filter at a temperature of 80-90° C., whereby membrane vesicles of the cell-derived biological membrane are formed.

7. The method of claim 4, further comprising combining the cell-derived biological membranes with the nanoshells at a ratio between 20:1 and 1:20.

8. The method of claim 7, further comprising co-extruding the cell-derived biological membranes and the nanoshells through a porous extruder filter at a temperature of 80-90° C.

9. Bio-nanoshells prepared according to the method of claim 4.

10. A method for delivering bio-nanoshells to target cells, comprising:
(a) exposing the target cells to bio-nanoshells, wherein each of the bio-nanoshells comprises a nanoshell having an exterior surface coated with a cell-derived biological membrane from a donor cell, wherein each of the cell-derived biological membranes comprises a phospholipid bilayer and an adhesion protein specific for one of the target cells; and
(b) binding the bio-nanoshells specifically to the target cells via the cell-derived biological membranes.

11. The method of claim 10, wherein the target cells are in a subject.

12. The method of claim 11, wherein the donor cells are from the subject.

13. The method of claim 11, further comprising administering the bio-nanoshell to the subject.

14. The method of claim 10, wherein the bio-nanoshells are photothermal, the method further comprising applying light to the bio-nanoshells.

15. The method of claim 14, further comprising emitting heat by the bio-nanoshells.

16. The method of claim 15, further comprising killing the target cells by the heat.

17. The method of claim 15, further comprising altering a function of the target cells by the heat.

18. The method of claim 15, wherein the target cells are in a subject suffering from a disease or condition, the method further comprising treating the disease or condition with the heat.

19. The method of claim 14, further comprising emitting photoluminescence or scattering light by the bio-nanoshells.

20. The method of claim 14, wherein the target cells are in a biological tissue, the method further comprising generating an acoustic wave by the biological tissue.
